(19) United States
(12) Patent Application Publication
(10) Pub. No.: US 2008/0177172 A1
(43) Pub. Date: Jul. 24, 2008

(54) Two-dimensional or three-dimensional imaging of a target region in a hollow organ

(76) Inventors: Matthias John, Nuremberg (DE); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/903,536

(22) Filed: Sep. 21, 2007

(30) Foreign Application Priority Data
Sep. 28, 2006 (DE) 10 2006 046 045.6

(51) Int. Cl. A61B 5/055

(52) U.S. Cl. 600/413; 382/131

(57) ABSTRACT

A two-dimensional or three-dimensional imaging of a target region in a hollow organ is provided. A two- or three-dimensional reconstruction image dataset is reconstructed from two-dimensional images from the inside of the hollow organ that are recorded by via a rotating image recording device and displayed, with images covering the entire target region being recorded during a partial rotation of the image recording device through a rotation angle.

### CROSS REFERENCE TO RELATED APPLICATIONS

This application claims priority of German application No. 102006046045.6 DE filed Sep. 28, 2006, which is incorporated by reference herein in its entirety.

### FIELD OF INVENTION

The invention relates to a method for the two-dimensional or three-dimensional imaging of a target region in a hollow organ, wherein a two- or three-dimensional reconstruction image dataset is reconstructed from two-dimensional images from the inside of the hollow organ that are recorded by means of a rotating image recording device and displayed.

### BACKGROUND OF INVENTION

In examinations or minimally invasive interventions in hollow organs, in particular in the region of the heart, medical instruments are used which include an image recording device. By means of the images of said image recording device it is aimed to examine a target region of a hollow organ or to monitor an intervention that is taking place therein. Since it has been customary in the past to display two-dimensional images of the image recording device immediately for this purpose, it has been proposed to reconstruct and display a plurality of two-dimensional images of the rotating image recording device in the form of a two- or three-dimensional reconstruction of the hollow organ.

Thus, for example, it is possible to produce a three-dimensional reconstruction image dataset of the entire hollow organ or a two-dimensional reconstruction image dataset of the surface of the hollow organ, for example, over the entire hollow organ after each complete revolution by means of an image recording device, for example an ultrasound device, rotating about the longitudinal axis of a medical instrument, for example a catheter. The reconstruction image dataset can be updated during the continuous rotation of the image recording device, whereby either the latter can be rotated independently by means of a micromotor or the catheter as a whole can be rotated.

### SUMMARY OF INVENTION

However, difficulties arise here if the images are to be displayed in realtime, in particular in the case of rhythmically moving hollow organs, in particular in the region of the heart, where only two-dimensional single images recorded within the same phase in different ECG cycles can be used for the reconstruction image dataset. The recording of the two-dimensional single images during a full rotation of the image recording device through 360° therefore takes a very long time which can extend for example over several ECG cycles. In particular during an intervention, however, a more frequent updating of the reconstruction image dataset is required.

A further disadvantage arises from the fact that in addition to the target region of interest, as a result of the recording of the complete hollow organ a multiplicity of image information is recorded which ultimately is irrelevant to the actual medical problem under consideration.

The object of the invention is therefore to specify a method by means of which it is made possible to provide, by comparison with the prior art, an improved and therefore faster reconstruction and hence updating as well as visualization of a reconstruction image dataset showing a target region of interest.

In order to achieve this object, it is inventively provided in a method of the type cited in the introduction that images covering the entire target region are recorded during a partial rotation of the image recording device through a rotation angle.

It is therefore provided according to the invention that with a correspondingly positioned and orientated image recording device, two-dimensional images will no longer be recorded during a complete revolution of the image recording device through 360°, but that instead this will take place only during a partial rotation through a specific rotation angle. Said rotation angle as well as the orientation (including, of course, also the line of vision of the image recording device) and the position are chosen in this case in such a way that even if recordings are taken only during a partial rotation, these images will be suitable for reconstructing the entire target region of interest therefrom. The two-dimensional images acquired during the partial rotation cover the target region. This means that advantageously fewer images can be recorded, thus saving time, or that the rotation can be performed more quickly overall. It is therefore possible by means of the method according to the invention to perform the image recording within a single heart cycle if necessary, with the result that ultimately a new, updated reconstruction image will be displayed after each heart cycle.

In order to obtain a regular recording and reconstruction as well as an up-to-date display, the method is performed iteratively with successive partial rotations. In particular the reconstruction and display advantageously take place in realtime, which means that as soon as new two-dimensional images have been recorded by the image recording device during the partial rotation, the reconstruction image dataset is updated.

In general, during the method according to the invention, the image recording device or the medical instrument, in particular the catheter, serving as its carrier is initially positioned and orientated in such a way that in the course of a partial rotation at a specific angular interval the target region can be recorded completely. In this case the rotation angle determines the angular interval accordingly in common with a line of vision of the image recording device, which either reproduces the starting point of the recording of the two-dimensional images, in which case the image recording device is then rotated further by the rotation angle during the image recording, or else can also reproduce the center of the corresponding circle segment, such that the partial rotation extends through half the rotation angle in both directions in each case. While the image recording device executes said partial rotation, its field of vision therefore sweeps the target region of interest in the hollow organ, thereby enabling two-dimensional images to be produced. In order to execute the partial rotation, the rotation of the image recording device should preferably be performed by a motor which is controlled for example by a control device, since by this means uniform speeds and a precise adherence to the angular interval are made possible.

The overall movement of the image recording device during and between individual updating image recording operations can proceed in different ways. First, it is possible that the image recording device continues to be rotated continuously through 360°, with recordings being taken only when sweeping the angular interval which defines the partial rotation. In this case it is then possible in particular for the image recording device to be stopped for example after each complete rotation in order to achieve synchronism with a movement cycle of the hollow organ that is to be recorded, in particular with the ECG cycle. In such a case, however, the angular speed of the image recording device can preferably be adjusted in such a way that a sweep of the target region is essentially performed in the same movement phase, in particular the same ECG phase, with the result that all the reconstruction image datasets correspond to the same phase. Furthermore a continuous rotation of the image recording device is then possible.

On the other hand it is, of course, also possible for the image recording device to perform only the partial rotation. For this purpose it can be moved back and forth for example successively between an angle marking the beginning of the angular interval and an angle marking the end of the angular interval, whereby images can be recorded only during movement in one direction or even during movement in both directions. For this purpose a corresponding configuration of the control device and the motor driving the rotation is then necessary.

If the field of vision or the recording area of the two-dimensional images can be characterized for example in terms of its shape as a trapezoid or circular ring segment, an image shape of this kind is also described as a “butterfly wing” because of the similarity of form. A recording of this kind of images during a partial rotation would then result in a “butterfly wing beat”, in other words two-dimensional images arranged sequentially in the direction of rotation and describing a certain three-dimensional volume. From a “butterfly wing beat” of this kind, a three-dimensional reconstruction of this volume or a two-dimensional reconstruction of a surface lying within this volume can then be computed and visualized in accordance with known methods.

In order to achieve a significant reduction in the number of two-dimensional images requiring to be recorded and thereby enable faster recording of images, it can be provided that a rotation angle less than 180°, in particular less than 90°, will be used. In particular, given suitable positioning and orientation of the image recording device, rotation angles less than 90°, in particular less than 60° even, are frequently already suitable for covering the target region of real interest, in which, for example, the intervention takes place or in which, for example, a lesion is suspected.

As already mentioned, a particularly high updating rate in the case of a recording in a region subject to the rhythmic movements of the heart will be advantageously increased if the ECG-triggered recording of a set of two-dimensional images used for the reconstruction is completed during a single heart cycle. This means in particular that it is possible, owing to the smaller number of images to be recorded, to record all the two-dimensional images necessary for the reconstruction already during a single heart phase in which no significant changes of the hollow organ due to the movement occur, with the result that it is made possible to update the displayed reconstruction image dataset at least after each heartbeat.

The positioning and orientation of the image recording device or of the medical instrument, in particular the catheter, serving as its carrier can be of great significance for the method. In order to enable same, it is important in particular to know the current position and orientation of the image recording device, for which purpose navigation systems are already known in the prior art which, with the aid of electromagnetic sensors mounted on a catheter tip for example, continuously determine the position and orientation of the medical instrument and hence also of the image recording device, which has a fixed geometric relationship with the catheter tip. It should be taken into account here that in the case of an image recording device which is rotated independently of the medical instrument, a means should be provided for establishing the current line of vision of the image recording device, i.e. its current angle of rotation. Within the scope of the method according to the invention a navigation system of said kind can be beneficially used.

Thus, it can be provided that a before-image dataset of the hollow organ, registered with the coordinate system of a navigation system for determining the position and orientation of the image recording device, is displayed, by means of which the target region is defined by a user. In this embodiment a before-image dataset representing the hollow organ including the possible target regions of interest is accordingly displayed to a user. In this before-image dataset it is now possible for a user to define a target region, for example by marking using suitable marking tools, which is then to be recorded by means of the partial rotation. As the navigation system and the before-image dataset are registered with one another, it is possible to position and orientate the image recording device such that the recording of the two-dimensional images can be performed and the target region is completely covered.

The registration process can be performed by means of essentially known methods; a landmark-based registration can be performed, for example. In this case the image recording device or the medical instrument serving as its carrier is guided under x-ray control to specific anatomically significant points. Examples of such points in the case of a cardiac examination or treatment are the mouth of the superior vena cava, the mouth of the inferior vena cava, the heart valves, etc. The catheter position is recorded by means of the navigation system at these anatomically significant points and stored for each of the significant points. In the before-image dataset, which in this case is preoperative, the same points are identified and the registration is performed on the basis of the data associated with the anatomically significant points.

Alternatively, image-based, in particular 3D-3D, registration methods can also be used. In this case a small number of two-dimensional images from which a three-dimensional volume can be reconstructed are used for the registration. Equally, it is possible to extract surfaces from image datasets of the image recording device and the before-image dataset by segmentation, whereby the registration is performed as “matching” of the two extracted surfaces. In this case it is not necessary to extract a complete surface: two so-called “point clouds”, made up of a small number of points which represent the surfaces, are also sufficient for performing a registration by minimizing the distance between the two point clouds.

Particularly advantageously, made possible as a result of the registration, the position and orientation of the image recording device and/or of a medical instrument serving as its carrier can also be displayed in the before-image dataset. The user then knows how and where the image recording device is currently located and can position and orientate the device manually if necessary so that the recording can be performed by partial rotation.

However, a first possibility of defining and subsequently recording the target region provides that from the definition of the target region which has been marked for example by a user in the before-image dataset, an ideal position and orientation of the image recording device will be determined and displayed in the before-image dataset, whereupon the image recording device will be guided automatically or with user support to the destination. In this case a computing device is provided which uses the data of the before-image dataset as well as the characteristics of the catheter in order to calculate at which position and orientation of the image recording device it is possible to make the fastest possible and yet qualitatively satisfactory recording of the target region by means of two-dimensional images during a partial rotation. This position and orientation are then also represented in the before-image dataset, in a different color for example, so that a user guiding the medical instrument serving as carrier of the image recording device can bring the medical instrument and hence the image recording device, since its position and orientation are likewise displayed to him, into the computed ideal position. In another embodiment of the method this can also take place automatically. In this embodiment the user must simply mark the target region in the before-image dataset, whereupon the necessary recording parameters, i.e. rotation angle, where appropriate depth of field, line of vision, orientation and position of the image recording device, are determined automatically.

In a particularly advantageous embodiment it is, however, provided that in addition to the position and orientation of the image recording device and/or of a medical instrument serving as its carrier, the field of vision of the image recording device is also represented in the before-image dataset, taking into account a set rotation angle and a set depth of field. In this case the user can establish solely by looking at the before-image dataset and the additional information represented therein, which region of the hollow organ he would record if he were to start the image recording using these parameters, which can be changed by the user himself. In the same way that he can also influence the position and orientation of the image recording device, the user is therefore able to set the rotation angle and/or the depth of field as well as possibly also quality parameters of the image recording device by means of an input device and to observe immediately how the field of vision of the image recording device changes. The current field of vision is updated in realtime based on the set parameters and the data of the navigation system, with the result that the user has an overview of his recording options. Alternatively or in addition it can also be provided in this context that a virtual image recording device and/or a virtual catheter serving as its carrier, as well as the field of vision of the virtual image recording device, are inserted in the before-image dataset. In an embodiment of this kind, even prior to the intervention or while the medical instrument has not yet reached the target region, the user can try out, as it were, in which position and orientation of the image recording device he can record which areas using which parameters. The data necessary for this can be stored in a computing device for example.

In both cases, i.e. both when the field of vision of the real image recording device or the field of vision of the virtual image recording device is inserted, it is possible for the target region to be defined on the basis of the representation of the field of vision. When guiding the catheter, the user is shown on the display at all times, for example in the form of a transparent overlay, which area he can record using the current parameters set by him. This can then be easily selected, via a confirmation control element for example, so that the target region is defined by the field of vision inserted in this instant. The recording can then start immediately and takes into account the parameters set by the user. In this way an easy-to-use method supporting the user with all the necessary information can be created which permits simple parameter adjustment, selection and guidance of the image recording device to the destination point as well as a subsequent fast, current recording and updating of a reconstruction representation. The user first suitably positions and orientates the medical instrument and hence the image recording device, then adjusts the image recording parameters and during the entire time can track to what extent the desired target region has been recorded.

Alternatively or in addition, a two-dimensional image or layer image, in particular a multiplanar reconstruction (MPR), can be selected from the before-image dataset for the purpose of defining the target region. Said two-dimensional image or layer image of the before-image dataset can serve for example as a central plane of the partial area to be reconstructed. The selected image accordingly specifies the orientation of the volume to be swept by the image recording device. In this case the image recording device is placed within the selected plane in such a way that its axis of rotation lies in the plane. This is possible without difficulty because of the representation of the image recording device or of the medical instrument serving as its carrier.

In addition to the position and orientation of the image recording device or of a medical instrument serving as its carrier, the position and/or orientation of a further medical instrument located in the hollow organ, in particular of a working catheter, can also be represented in the before-image dataset. If an intervention, an ablation for example, is carried out, the image recording device can accordingly be positioned in such a way that the further medical instrument is also included in its field of vision, with the result that the progress of the intervention can be observed.

In this case a plurality of image datasets which show the hollow organ can be selected as the before-image dataset. On the one hand the before-image dataset can be generated by means of a reconstruction during a full rotation of recorded two-dimensional images of the image recording device. The before-image dataset is recorded here by means of the image recording device itself, possibly with a lower quality, in order to be able to provide a good overview of the hollow organ. A further image recording modality is not necessary in this instance. The registration is also easy to perform in this case.

Alternatively, a preoperative image dataset, in particular a computed tomography, magnetic resonance or rotation angiography image dataset, can also be used as the before-image dataset. Preoperative before-image datasets of this kind also provide a good overview and can also make a supporting contribution already during the guidance of the medical instrument serving as carrier of the image recording device into the target region. Moreover, irregularities that need to be treated or investigated more closely are often to be recognized therein.

If such a preoperative before-image dataset is used, the reconstruction of the two-dimensional reconstruction image dataset can be performed taking into account a two-dimensional image, in particular a curved one, selected by the user in the before-image dataset. A curved, two-dimensional image of this kind can be, for example, what is referred to as a “curved MPR” which shows the wall or a specific wall area of the hollow organ. Said image determines the surface which is to be reconstructed from the two-dimensional images of the image recording device in the angular interval and displayed. In this way the user can advantageously select already in the before-image dataset which section of the target region is to be reconstructed two-dimensionally.

It is also particularly advantageous if, in the case of ECG-triggered recordings, the two-dimensional images of the partial rotation are recorded during the same ECG phase as the before-image dataset. Hollow organs frequently change their shape to a major degree, depending on the heart phase. Accordingly it can happen that, for example, a tissue section lying inside the target region during the phase of the recording of the before-image dataset is no longer located within the target region at another point in time. It therefore makes sense, in order to be able to specify the target region as precisely as possible in relation to the hollow organ, to perform the triggering during the same ECG phase.

In addition, the reconstruction image dataset can also be represented in the before-image dataset, for example by overlaying the corresponding information. The user then only has to look at one image representation.

An ultrasound device or an OCT (Optical Coherence Tomography) device, which are particularly suitable for recording such two-dimensional images, can be used for example as the image recording device.

In addition, the invention also relates to a medical examination and treatment system, comprising a medical instrument which can be introduced into a hollow organ and has a rotatable image recording device, in particular an ultrasound or OCT device, as well as a control device, said control device being embodied for controlling the image recording device in such a way that the latter executes a partial rotation through a specific rotation angle.

In this way it is not only possible to make the binary selection “rotation” or “no rotation”; rather, predefined partial rotations can also be performed. During a partial rotation of this kind a specific target region can then be recorded. In particular, the image recording device can also be embodied to be rotatable in both directions. It is then possible to move the image recording device forward and back again through a specific rotation angle for example.

In addition an actuator for tilting a part of the instrument tip including the image recording device and/or the image recording device against the central axis of the instrument tip can also be provided. This finally enables a different perspective to be set in that the image recording device is tilted in particular against the axis of rotation. It is also possible in this embodiment to align the image recording device parallel to a wall of the hollow organ for example. In this arrangement the actuator can be embodied for example as a Bowden cable.

A system of this kind can advantageously be used for performing the method according to the invention, since images can be recorded without difficulty during the partial rotation owing to the control capability.

### BRIEF DESCRIPTION OF THE DRAWINGS

Further advantages and details of the present invention will emerge from the exemplary embodiments described in the following as well as with reference to the drawings, in which:

FIG. 1 shows a medical examination and treatment system according to the invention,

FIG. 2A is a representation of the outline of a two-dimensional image which can be recorded by means of the image recording device,

FIG. 2B shows the field of vision obtained when recording a plurality of such two-dimensional images during a partial rotation,

FIG. 3 is a flowchart of the inventive method according to a first exemplary embodiment,

FIG. 4 is a representation of the before-image dataset with additional information in the case of the first exemplary embodiment,

FIG. 5 is a flowchart of the inventive method according to a second exemplary embodiment, and

FIG. 6 is a representation of the before-image dataset with additional information in the case of the second exemplary embodiment.

### DETAILED DESCRIPTION OF INVENTION

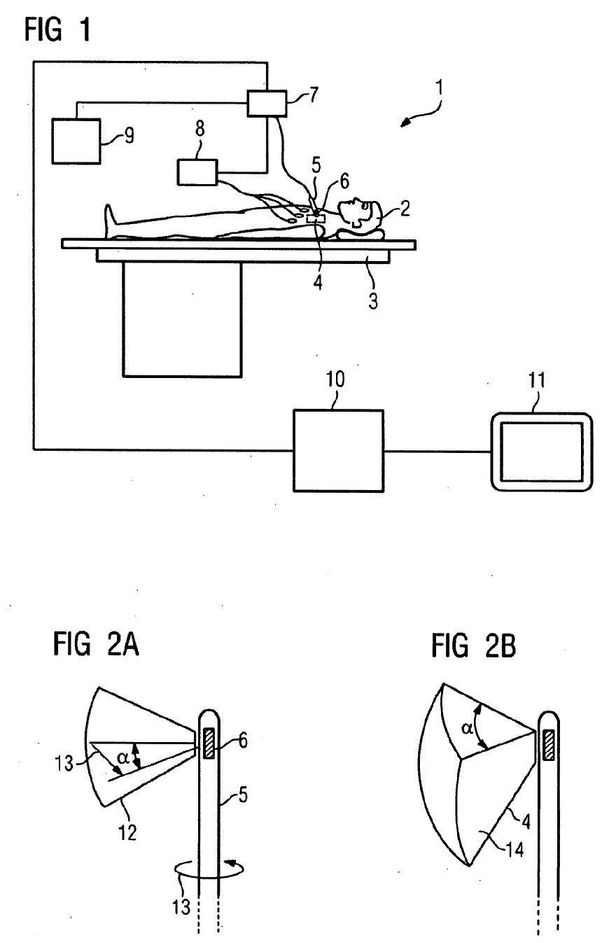

FIG. 1 shows a medical examination or treatment system 1. In this example a patient 2 is lying on a positioning table 3, the patient including a target region 4 that is to be recorded, in this case in particular in the cardial region, into which a catheter 5, comprising a rotatably disposed image recording device 6, has been introduced. The catheter 5 and the image recording device 6 are controlled accordingly by means of a catheter control device 7, though a guiding of the catheter by hand is also possible. An ECG device 8 records the ECG of the patient 2. The ECG device 8 sends its data to the catheter control device 7 so that a triggered recording of two-dimensional images by means of the image recording device 6, in this example an ultrasound device, can be performed. The position and orientation of the image recording device of the catheter 5 can be determined at any time by means of a navigation system indicated by the reference numeral 9. The control device 7 communicates with a computing device 10 in which recorded images can be processed and by which parameters input on the user side can be transmitted to the control device 7. The system 1 additionally comprises a display device 11, in this case a monitor, for displaying image datasets.

The control device 7 and the rotation unit (not shown here) of the image recording device 6 are therein embodied in such a way that the image recording device is able to execute a partial rotation through a specific rotation angle, whereby two-dimensional images are recorded at a predetermined frequency. Moreover, the image recording device 6 can be rotated in both directions, with the result that it is possible for example to move the image recording device 6 back and forth repeatedly through the rotation angle for the purpose of repeatedly scanning a target region 4. It is also conceivable, of course, that the image recording device 6 is rotated in one direction only, with an image being recorded only at a specific angular interval.

From the two-dimensional images recorded during a partial rotation of this kind it is possible to acquire a two- or three-dimensional reconstruction image dataset by means of the computing device 10. The entire field of vision from which the reconstruction image dataset is acquired results from the shape of a two-dimensional image and the rotation angle or angular interval. This is illustrated in more detail by an example in FIGS. 2A and 2B.

FIG. 2A shows a representation of the outline 12 of a two-dimensional image which can be recorded by means of the image recording device 6 in a specific angular position. It has a roughly trapezoidal shape, often also described by the term “butterfly wing”.

If the image recording device 6 is now rotated through a rotation angle α, as shown in FIG. 2A by means of the arrows 13, with two-dimensional images being recorded simultaneously at a specific frequency, a reconstruction image dataset which describes the volume 14 shown in FIG. 2B can be derived therefrom. Said volume 14 corresponds to the target region 4. Similarly to the term “butterfly wing”, cf. 2A, its shape can be described as a “butterfly wing beat”.

If only a partial rotation of this kind is necessary for recording the target region 4, fewer images in total are required and the recording can be performed more quickly, in particular even during one ECG phase of an ECG cycle. It is, however, also possible to record the images during two succeeding ECG cycles given corresponding triggering, since the control device 7 can control the rotation unit accordingly.

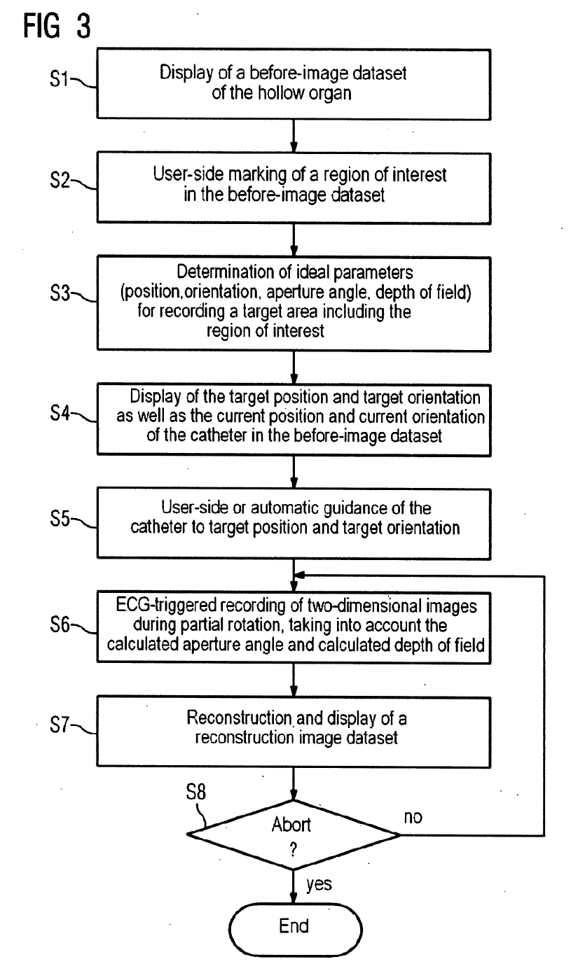

FIG. 3 shows the flowchart of the inventive method according to a first embodiment. First, in step S1, the user is presented with a before-image dataset of the hollow organ on the display. A before-image dataset of this kind may have been recorded by means of the image recording device 6 itself, for example through a 360° rotation, a recording of lower quality being acceptable in the case of such an overview image dataset. Another possibility for a before-image dataset is a preoperatively recorded image dataset, for example a computed tomography image dataset, a magnetic resonance image dataset or a rotation angiography dataset. The before-image dataset is a three-dimensional image dataset which can be visualized in various, essentially known ways, for example as an “on-the-fly” visualization.

The before-image dataset is registered with the coordinate system of the navigation system 9, with the result that the position and orientation of the catheter 5 as well as of the image recording device 6 and also of the corresponding recordable two-dimensional images are also known in the before-image dataset. The registration required for this can be effected for example by way of anatomical landmarks, but also by means of known 3D-3D registration methods.

In the representation of the hollow organ in the before-image dataset, it is now possible, in step S2, for a user to mark a region of interest (ROI), which operation can be performed via an input device associated with the computing device 10. Methods for marking a three-dimensional region in an image representation are widely known, so they do not need to be discussed in more detail here.

In step S3, the computing device 10 determines ideal parameters for recording a target area including the region of interest, said parameters including the position and orientation of the image recording device 6 as well as the rotation angle and where appropriate the depth of field. In this case the target area encloses the region of interest as closely as possible so that an absolute minimum of unwanted information is recorded. Additionally taken into account here are quality aspects which can also be specified by the user if necessary.

It should be noted at this juncture that a virtual catheter comprising a virtual image recording device and its field of vision can also be inserted into the before-image dataset in the course of a simulation or modeling. In this case a user can change the position and orientation of the virtual image recording device as well as the rotation angle and the depth of field in order to adjust the field of vision according to his requirements. It is thus possible to specify a target region as well as the corresponding parameters directly on the user side without actually moving the catheter 5 in the hollow organ.

In any case this is followed by step S4, in which the target position and target orientation as well as the current position and current orientation of the catheter 5 are displayed in the before-image dataset. The user therefore sees immediately how the catheter 5 and the image recording device 6 are currently located in relation to the target position and target orientation.

Based thereon, in step S5 the user himself can move the catheter to the target position and target orientation, though this catheter guidance can also be performed automatically. The user-side guidance is implemented easily owing to the image support described by step S4.

Assuming the target position and target orientation have been reached, in step S6 the ECG-triggered recording of two-dimensional images begins, while the catheter 5 performs a partial rotation through the specific rotation angle which finally defines the angular interval at which the recordings of the two-dimensional images are to be made. In addition, the image recording device 6 is adjusted according to the calculated depth of field. By this means two-dimensional images which cover the entire target region are recorded during the partial rotation. In this case fewer images are recorded than in the case of a complete revolution. The ECG triggering advantageously corresponds to that of the before-image dataset so that the region of interest will contain the required details. Preferably only one heart cycle is necessary for recording a full set of two-dimensional images.

After a complete set of two-dimensional images has been recorded, a reconstruction image dataset in two or three dimensions is calculated therefrom by the computing device 10 and displayed on the display device 11. This takes place in step S7. In this case it is possible both to display the reconstruction image dataset as an additional image and to display it in the before-image dataset.

In step S8, finally, a check is made to determine whether an abort condition, an instruction from the user for example, is present. If this is not the case, steps S6 and S7 are repeated so that the user, thanks to the fast recording capability, always receives an up-to-date reconstruction image dataset and can thus track changes in realtime.

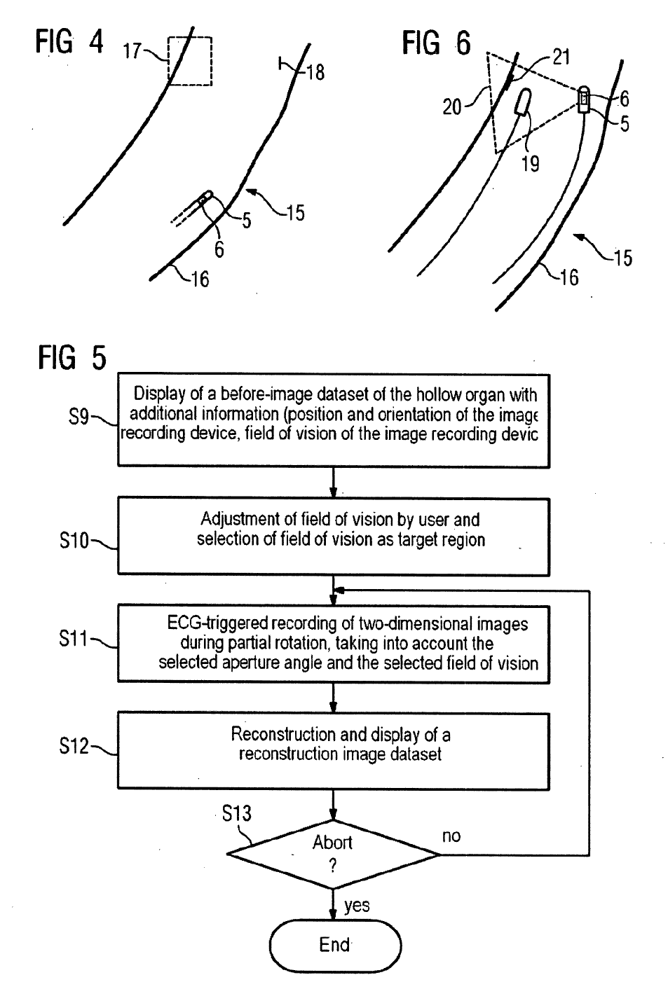

FIG. 4 illustrates by way of example a representation of the before-image dataset 15 which shows the hollow organ 16. Also displayed is the region of interest 17 marked by the user, together with the target position and target orientation 18 required to record it and to be assumed by the image recording device 6. Also shown are the position of the catheter 5 comprising the image recording device 6 and the corresponding orientation.

With the aid of such a visualization it is easy for a user to move the catheter 5 and therefore the image recording device 6 to the target position and into the target orientation, since the position and orientation of the catheter 5 are constantly being determined and updated by the navigation system 9 and incorporated in the visualization.

FIG. 5 shows the flowchart of the inventive method according to a second exemplary embodiment. There, in step S9, a before-image dataset of the hollow organ, albeit with various additional information, is displayed once again. The before-image dataset can again be recorded using the image recording device 6 itself, but may also be a preoperative image dataset. Moreover, the coordinate system of the before-image dataset is again, as already explained above, registered with the coordinate system of the navigation system 9.

By this means it is also possible to display the position and orientation of the catheter 5 and hence the image recording device 6 in the before-image dataset. In addition, however, the field of vision of the image recording device 6 in the case of the current parameters is also inserted. The field of vision of the image recording device 6 is determined not only from the position and orientation of the image recording device 6, but in particular also from a specific rotation angle and where appropriate, if this can be set, the depth of field.

If an intervention with a further catheter, for example a working catheter for the purpose of ablation, is to be performed, the position of said working catheter and if necessary its orientation can also advantageously be represented in the before-image dataset. It is then immediately visible to a user whether the working catheter and/or the region to be treated are located within the field of vision of the image recording device 6 with the currently set parameters.

In step S10, a user can then adjust the field of vision according to his requirements. For this purpose he can for example alter the position and orientation of the image recording device 6. Using suitable input means it is, however, also possible to adjust the rotation angle and if necessary the depth of field accordingly. Each change to this information is immediately represented in the before-image dataset. In this way the user can adjust the field of vision easily according to his requirements. If the field of vision corresponds to the region which the user would like to have recorded and displayed, he can start the recording via the input device and thereby specify the current field of vision as the target region.

In this exemplary embodiment the two-dimensional images are recorded, reconstructed and displayed in steps S11 and S12 in an analogous manner to the first exemplary embodiment.

Also in an analogous manner to the first exemplary embodiment, a check is made in step S13 to determine whether an abort condition is present, for example whether an intervention has already been terminated. If no abort condition is present, steps S11 and S12 are repeated in this case too, with the result that the user receives an up-to-date representation.

FIG. 6 shows a representation of the before-image dataset with additional information in the case of the second exemplary embodiment. The before-image dataset 15 again contains image data of the hollow organ 16. Similarly, the current position and orientation of the catheter 5 and the image recording device 6 are shown. In addition the user can also derive the position and orientation of a working catheter 19 from the representation.

The field of vision 20 of the image recording device 6 is also inserted in the representation, with both the working catheter 19 and a lesion 21 requiring treatment being included within the field of vision 20, i.e. the recording area of the image recording device 6. On the basis of this representation a user can now adjust the field of vision 20 according to his requirements by changing the position and orientation of the image recording device 6 and adjusting the rotation angle and depth of field parameters, in order thereafter to start the recording with said field of vision 20 as the target region.

The possibilities for selecting the target region and determining the parameters as shown in the two exemplary embodiments are not exhaustive. Thus, for example, it is also conceivable that a sectional or layer image is determined in the before-image dataset, which image is intended to form the central plane of the target region and consequently at least partially defines the orientation of the image recording device. The corresponding layer or plane can then, for example, also be inserted in the representation of the before-image dataset, with the result that the catheter and hence the image recording device now only need to be suitably placed within said plane.

The reconstruction image dataset can be represented in a known manner. However, it is also possible in particular within the scope of the invention to select from within the before-image dataset an, in particular curved, two-dimensional image or a surface area which, in a two-dimensional reconstruction, indicates the surface area which is to be reconstructed. In this case, what is referred to as a “curved MPR” springs to mind as an example.

An OCT device could also be provided as the image recording device. This image recording method also permits two-dimensional images to be recorded, from which a three-dimensional reconstruction volume can be computed.

It can additionally be provided that an actuator is present for the purpose of tilting a part of the instrument tip, in particular catheter tip, comprising the image recording device and/or the image recording device against the central axis of the instrument tip, in particular catheter tip. Then it is possible, for example, to tilt the image recording device against the axis of rotation or to orientate it such that it is aligned in parallel with a wall of the hollow organ.

1.-24. (canceled)

25. A method for two-dimensional or three-dimensional imaging of a target region of interest in a hollow organ, comprising:
recording a reconstruction image dataset from two-dimensional images from the inside of the hollow organ during a partial rotation of a single rotating image recording device through a rotation angle, the rotation angle less than 360°, images of the entire target region are recorded during the partial rotation; and
displaying the reconstruction image dataset.

26. The method as claimed in claim 25, wherein the rotation angle is less than 90°.

27. The method as claimed in claim 25, wherein the recording and displaying are performed repeatedly in successive partial rotations and performed in realtime.

28. The method as claimed in claim 27, wherein an ECG-triggered recording of a set of two-dimensional images used for the recoding is performed during a single heart cycle.

29. A method for two-dimensional or three-dimensional imaging of a target region of interest in a hollow organ, comprising:
displaying a before-image dataset of the hollow organ registered with the coordinate system of a navigation system for determining a position and an orientation of the image recording device;
recording a reconstruction image dataset in real time from two-dimensional images from the inside of the hollow organ during a partial rotation of a single rotating image recording device through a rotation angle, the rotation angle less than 360°, images of the entire target region are recorded during the partial rotation; and
displaying the reconstruction image dataset,
wherein the target region is defined by a user using the display of the before-image dataset.

30. The method as claimed in claim 29, wherein an ideal position and an ideal orientation of the image recording device are determined from the definition of the target region, whereupon the image recording device is guided to the target location automatically or with user support.

31. The method as claimed in claim 30, wherein a field of vision of the image recording device is represented in the before-image dataset taking into account the rotation angle and a set depth of field.

32. The method as claimed in claim 31, wherein at least the rotation angle or the depth of field are changeable via the user.

33. The method as claimed in claim 31, wherein the target region is defined based on the representation of the field of vision.

34. The method as claimed in claim 29, wherein a multiplanar reconstruction is selected from the before-image dataset for defining the target region.

35. The method as claimed in claim 34, wherein the plane of the selected image or layer image serves as a central plane of the target region.

36. The method as claimed in claim 29, wherein the before-image dataset is generated via a reconstruction of two-dimensional images of the image recording device recorded during a full rotation.

37. The method as claimed in claim 29, a preoperative image dataset selected from the group consisting of computed tomography, magnetic resonance and rotation angiography image dataset is used as the before-image dataset.

38. The method as claimed in claim 37, wherein the reconstruction of the two-dimensional reconstruction image dataset takes into account a two-dimensional image selected by the user in the before-image dataset.

39. The method as claimed in claim 29,
wherein the recordings are triggered via an ECG, and
wherein the two-dimensional images of the partial rotation are recorded during the same ECG phase as the before-image dataset.

40. The method as claimed in claim 29, wherein the reconstruction image dataset is represented in the before-image dataset.

41. The method as claimed in claim 29, wherein an ultrasound device or an OCT device is used as the image recording device.

42. A medical examination and treatment system, comprising:
a catheter to be introduced into a hollow organ and has a single rotatable image recording device; and
a control device that controlling the image recording device such that the image recoding device executes a partial rotation through a specific rotation angle, the specific-rotation angle less than 360°.

43. The system as claimed in claim 42, wherein the image recording device is embodied to be rotatable in both directions.

44. The system as claimed in claim 42, further comprising an actuator for tilting a part of the catheter comprising the image recording device.
